(19) United States
(12) Patent Application Publication
(10) Pub. No.: US 2011/0224641 A1
(43) Pub. Date: Sep. 15, 2011

(54) Magnetic conductive recipient

(76) Inventor: Jean Brault, Cowansville (CA)

(21) Appl. No.: 12/661,281

(22) Filed: Mar. 15, 2010

(51) Int. Cl. A61M 37/00; A61N 2/00; A61B 5/055

(52) U.S. Cl. 604/500; 600/9; 600/410

(57) ABSTRACT

A magnetic conductive recipient comprising: magnetic field via magnetized water. This process provides means for drug delivery in a molecular state, control of blood circulation, control of internal and/or external cell organism functions, including MWRI (magnetic water resonance imaging).

### BACKGROUND

In the ongoing effort to better control administered drugs to a patient and following the process to reach the desired outcome during and after administration. Much as been aimed at controlling the quantities that are administered. Some examples of this are: Zero-order drug delivery using infusion pumps, time release drug patches, or the use of polymers such as described with U.S. Pat. No. 7,187,969 B2 entitled “ELECTROACTIVE PORE” and included here as reference. However there as been less attempt at controlling the drugs once administered. Some exceptions to this, such as time release capsules, have some control after administration, in relation to time and quantity. However this does not address control over the drugs orientation once they have been administered, which leaves a blind spot as to the extent of their effectiveness. One example of this; is blood circulation, and the outcome this may have on the drugs distribution, where both benefits and repercussions may occur. For example: when the initial target was a specific organ or cell(s), however neighboring organs, or cells are effected and/or contaminated by the administered drugs, brought on by the process of blood circulation. With the present embodiment, a patient is placed into a magnetic conductive recipient containing magnetized water that is in contact with the patient's skin wherein, drugs distribution and speed at which the drugs are distributed throughout the body, are both controlled after the drugs are administered, via: the magnetized water, that provides control over blood circulation. Furthermore theses processes may be monitored via MWRI (magnetized water resonance imaging), using drug dye, and will become more apparent in the summary, and claims.

### SUMMARY

A method, and apparatus, wherein any organism(s) including: a human(s), or animal(s), or cell(s), plant(s), DNA, RNA, clone organism(s), organic organism(s) and/or transgenic organism(s) is placed within a magnetic conductive recipient containing magnetized water. And in this process, creating a powerful new tool, having magnetic field control, over the magnetized water, via the magnetic conductive recipient. For example: wherein a human having been administered therapeutic agents: Control is achieved of the administered drugs distribution throughout the body, by controlling the blood circulation throughout the body, that is composed mainly of water, as described in (The effect of water drinking on the blood composition of human subjects in relation to diuresis), F. H. Smirk J. Physiol. VOL 78(2): 127-146) May 23, 1933 London ENGLAND; and included here as reference.

Herein the magnetic field is extended via the patient's blood, and controlled via the magnetic conductive recipient. Other benefits include: molecular drug delivery, blood filtering, control of blood flow, free radical extraction, pain control, reducing inflammation, to name some. Magnetic field via magnetized water can perform deep penetrating, and absorption process, via blood circulation. Furthermore providing MWRI (magnetized water resonance imaging), which distinguishes it from conventional MRI (magnetic resonance imaging) methods; where MWRI can reach at angles that is unheard of with conventional MRI methods. Taken a human body for instance. Magnetized water surrounds the body, and molds to every possible angle, by being in direct contact with the patients skin. Where conventional MRI as very limited control to reach angles in comparison. The consequence is distortion, a well known to the art fact, that is not adequately solved, whether it be MRI using coil, or open room MRI, both well known to the art.

Other methods and materials for drug delivery that may be used, in single, or in combination via the magnetic conductive recipient, are for example: pulsation, water agitation, to name some. Further uses with, MWRI comprises: SQUID (superconducting quantum interference device); that enable's magnetic fields operating conditions below 1 Telsa.

Further characteristics and advantages will become apparent from the detailed description, illustrated by non-limitative example.

### BRIEF DESCRIPTION OF THE DRAWINGS

FIG. 1 is a block diagram showing an ensemble of a system having a magnetic conductive recipient apparatus according to one embodiment of the present invention.

FIG. 2 is a diagram showing a detailed example of the structure of a magnetic conductive recipient apparatus to FIG. 1.

FIG. 3 is an exploded cross-sectional view of a cell magnetic conductive recipient unit according to another embodiment of the present invention.

FIG. 4 shows an above view of the magnetic conductive recipient for cells to FIG. 3, without the cover.

FIG. 5 shows an above view of the magnetic conductive recipient cover for cells to FIG. 3.

### DETAILED DESCRIPTION OF THE INVENTION

The present invention is described with reference to the drawings.

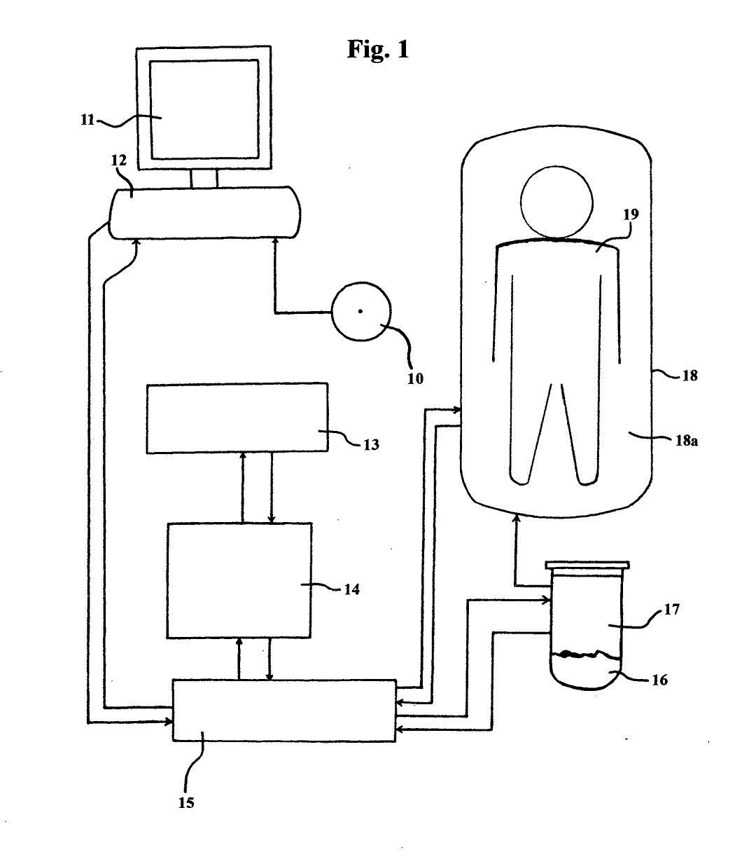

FIG. 1, is a block diagram showing an ensemble of a system via several units, wherein a magnetic conductive recipient apparatus for humans 18 is the center thereof. The magnetic conductive recipient 18 emits a magnetic field used for therapeutic treatments via methods, and means that comprises: software(s) 10, installed on a computer 12 having a computer monitor 11, are in turn connected to a control sequence transmitter receiver unit 15; that control a power supply 13, where electric current is controlled via a current regulator 14 before reaching the magnetic conductive recipient 18. This activates the magnetic field inside the magnetic conductive recipient 18; and in the process magnetizing the water 18b contained in the magnetic conductive recipient 18, shown surrounding a human organism 19. Means are provided for drug delivery (in a molecular state), via: the extraction process 17, wherein

Molecules deriving from substances 16 are extracted and transferred via the magnetic conductive recipient(s) 18. The extraction process 17 may take many forms, that comprises: steam distillation, cloud point extraction, supercritical carbon dioxide extraction to name some methods.

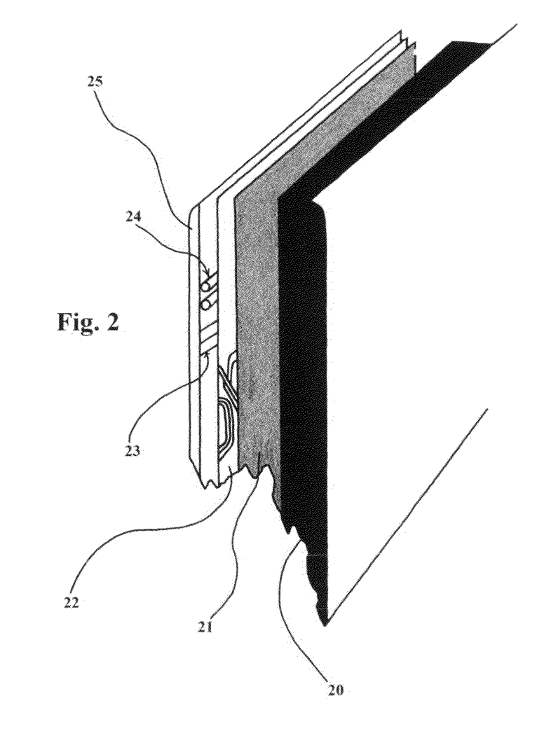

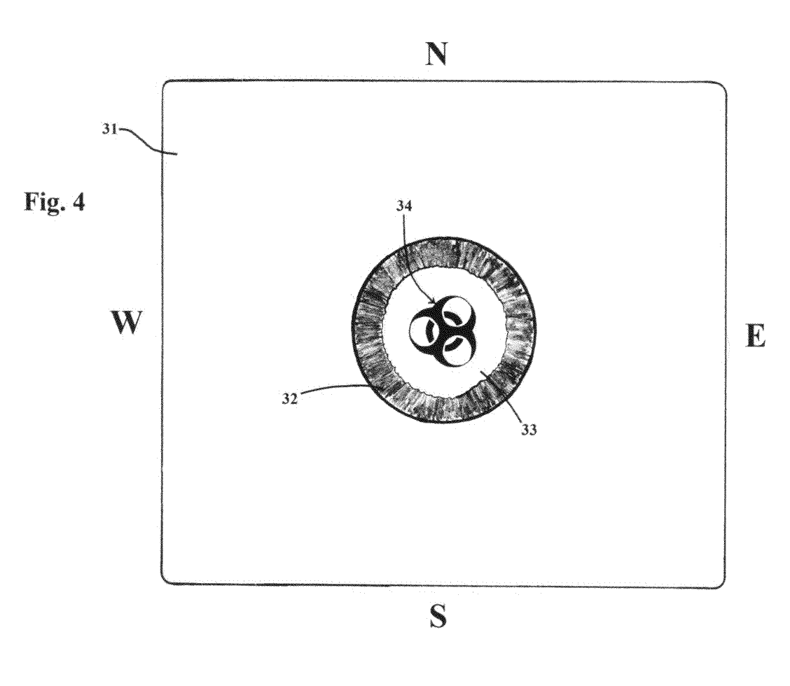

FIG. 2, shows the structural composition of a magnetic conductive recipient according to the present embodiment, wherein the exterior layer is a magnetic barrier layer 20, preventing exterior frequencies, exterior currents, and exterior magnetic fields, from penetrating, or interfering with the prescribed functions of the present embodiment. Shown in FIG. 2, is a container layer 21, wherein comprises carbon dioxide gas coolant, serving as a means for maintaining an adequate temperature within the magnetic conductive recipient structure FIG. 2, wherein tubing 24 serves to circulate carbon dioxide coolant from the container layer 21, throughout the structure of the magnetic conductive recipient. Super conductive wire 23, serves as transport means for drug delivery (in a molecular state) deriving from the extraction process 17 (FIG. 1), via the magnetic conductive materials layer 25; in turn via the magnetized water 18b (FIG. 1), and magnetized water 33 (FIG. 4) before reaching an organism in contact with the magnetized water, according to embodiments of the present invention. The magnetic conductive materials layer 25 is activated and controlled via circuit board panel(s) of layer 22. in turn these components control the magnetized water 18b (FIG. 1), and the magnetized water 33 as shown in (FIG. 4).

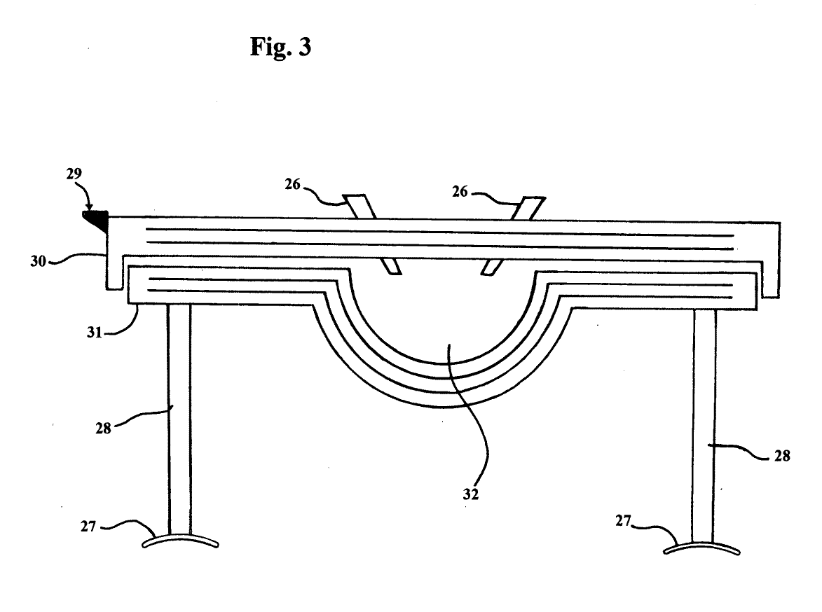

FIG. 3, shows a magnetic conductive recipient for cells 31, having support columns 28, that rest upon leveling mounts 27. A central cavity 32, containing magnetized water 33 as shown in (FIG. 4); herein the cells are placed. Furthermore the magnetic conductive recipient for cells 31, as a magnetic conductive recipient cover for cells 30, wherein conductive contact is made between 30 and 31, when 30 is placed onto 31. All other conductive connections that comprises: electrical power, sequence commands, drug delivery (in a molecular state) deriving from super conductive wire 23 (FIG. 2), via the extraction process 17 (FIG. 1); all connect via: port 29. Two multiple purpose tubes 26, traverse through the above exterior, of the magnetic conductive recipient cover for cells 30, into the central cavity 32, immediately above 32. Multiple purpose tubes 26 serve as means for introducing cells, as an entry for surgical tools, needles, and water replenishing means for central cavity 32, and as overflow means of central cavity 32, to name some.

FIG. 4, shows the magnetic conductive recipient for cells 31 (FIG. 3), and the central cavity 32 (FIG. 3) viewed from above, where a cell organism 34, is in direct contact with magnetized water 33. When the magnetic conductive cover for cells 30 (FIG. 3) (not shown) in FIG. 4, is joined with the magnetic conductive recipient for cells 31 (FIG. 3), as shown and described to claim 3; and is activated via means described to (FIG. 1). Multiple directional control is achieved, simultaneously (in every direction), as illustrated in FIG. 4, by: N=North, S=South, W=West, E=East. In turn spreading the process as described to claim 4, via the magnetized water 33, internally and externally controlling the cell organism 34, within the central cavity 32. Herein the described processes to FIG. 4 are in accordance to inverse square law

$$( \frac{S}{4 π {r}^{2}} = 1 ) .$$

More on the methods to FIG. 4, are to be found in the claims of the present embodiment.

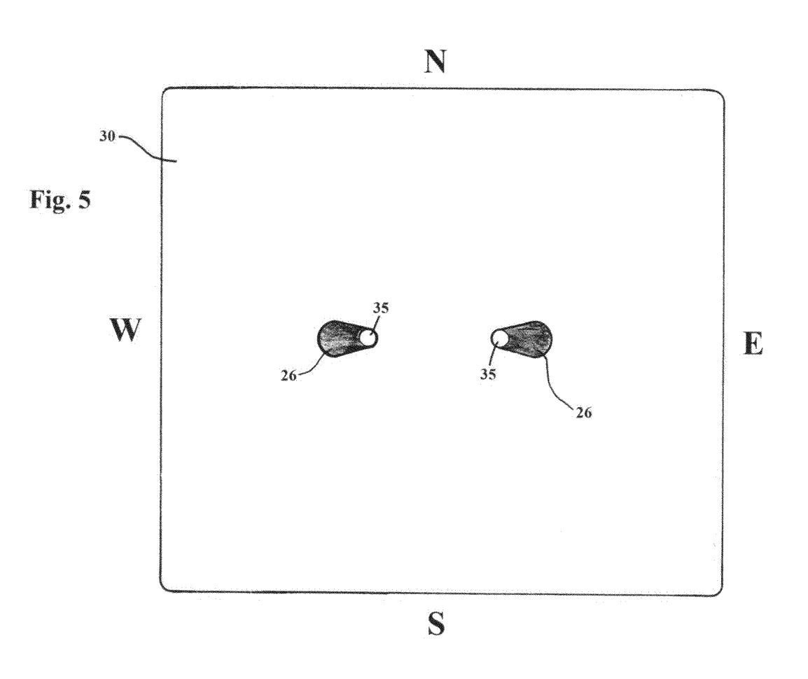

FIG. 5, shows an above view of the magnetic conductive recipient cover for cells 30 (FIG. 3), with an interior view of the multipurpose tubes 26 (FIG. 3). Further shows the insertion holes 35, within the multipurpose tubes 26 (FIG. 3).

Although particular embodiments of the invention herein have been described, it is not limited to this description. It is therefore to be understood that numerous modifications may be made to the embodiments without departing from the spirit and scope of the present invention. Other embodiments are to be found in the claims.

1. A method and apparatus wherein Any Organism(s) comprises: a human, or animal, or cell(s), plant(s), DNA, and/or RNA, clone organism(s), organic organism(s), and/or transgenic organism(s) but not limited to, is placed within a magnetic conductive recipient preferably containing: magnetized water; wherein, said organism(s) to claim 1, receives various therapeutic treatments, applications, according to the needs and type of organism; wherein said organism(s) contains blood (human blood is known to contain near 70% or more of water), comprises: control over distribution of drug delivery after administration, via: said magnetized water to claim 1, which surrounds said organism(s) to claim 1, wherein the magnetic field created via said magnetic conductive recipient to claim 1, enables said magnetized water to control blood circulation, but not limited to, in turn; providing control of the administered drugs via: North, South, East, West directions, in one or more directions simultaneously, and further comprises: overlapping of the boundaries of said North, South, East, West directions, consistent with Inverse Square Law
$$( \frac{S}{4 π {r}^{2}} = 1 ) ;$$
yet further comprises: blood filtering, pain control, scar healing, reducing of inflammation, but not limited to; said magnetic conductive recipient to claim 1, is controlled via:
software, installed on a computer having a computer monitor, in turn connected to a control sequence transmitter receiver unit, that control a power supply (that functions with AC or DC power, but not limited to), wherein, electric current is controlled via an electric current regulator, before reaching said magnetic conductive recipient; yet further comprises: remote receiver, transmitter signaling(s) means, (for one or more executions, and/or components to claim 1), (programmed software), that can simulate executions and/or said components to claim 1, but not limited to;
remaining with the present embodiment; however; said magnetic conductive recipient to claim 1 is not limited to containing said magnetized water to claim 1, therefore further comprises: water, steam, magnetic field, magnetic fabric, smart gels (responsive and non responsive gel), polymeric gels, magnetic gels, super paramagnetic gels, ferrogels, antiferromagnetic gels, oxygen, and/or other gas forms; substances, which comprises: nutrients, minerals, compounds, organic compounds, transgenic compounds, hormones, vitamins, herbs, amino acids, enzymes, drugs, drug dye, therapeutic agents, or a combination thereof, but not limited to; said substances to claim 1 will be termed as such, for obvious, and practical reasons; said substances being in the form of solid, liquid, or in a molecular state, or a combination thereof; said molecules to claim 1, which comprises: electrons, protons, ions, neutrons, atoms, or a combination thereof, but not limited to, will be termed as such for obvious and practical reasons; preferably said substances to claim 1 contained within said magnetic conductive recipient to claim 1, are in said molecular state, but not limited to;
said organism(s) to claim 1, are key elements, that determines the shape, size, material(s), and functions, of said magnetic conductive recipient to claim 1;
preferably, said conductive recipient to claim 1, for treating said human to claim 1, as the shape of a bath, but not limited to; however, wherein an animal, a (Horse) for instance: then, the shape of said magnetic conductive recipient to claim 1, will be in adequate proportion for said horse, said magnetized water to claim 1, and substances to claim 1, but not limited to; wherein said organism(s) to claim 1, are said cell(s) to claim 1; then again, said magnetic conductive recipient to claim 1, will yet have a different shape; therefore remaining with the present embodiment, further comprises: a shower, sauna, Jacuzzi, pool, aquarium, hydroponic container, green house, bag, vial, box, or any other shape, of container, or room, having, a cover or not; but not limited to;
wherein said organisms to claim 1, being said human; said magnetic conductive recipient, comprises: a drain(s), hot, and cold water inlet(s) and/or outlet(s), water temperature control unit, flow meter, a water filtering unit, or filtering system; these items, operate manually, or automated via: software, and or electrical components; further comprises: remote transmitter means; a cleaning system(s) via germicidal lamp(s), ultra sound, laser, infra red laser, pressure, pulsation, steam, water, or a combination thereof, but not limited to; said magnetic conductive recipient yet further comprises: raising and lowering means via: mechanical parts, that operate manually or via electric motor, having remote receiver transmitter means; said magnetic conductive recipient as mobility via: caster wheels, total lock caster wheels, swivel caster wheels, or via a combination thereof, but not limited to.

2. Wherein said organisms to claim 1, being said cell(s) to claim 1, of any said organism(s) to claim 1, comprises: control of (internal and/or external) functions, of said cell(s) to claim 1, and further comprises: means to control (internal and/or external) functions of mitochondrial contained within said cell(s) to claim 2, but not limited to.

3. Control over distribution, of said drug delivery to claim 1, after, said drug delivery via: blood circulation, but not limited to, wherein said organism to claim 1, is human, but not limited to; via: means to claim 1, but not limited to, in turn via: said magnetic conductive recipient to claim 1, via: magnetized water to claim 1, but not limited to;
further means for drug delivery to claim 3, comprises: via intravenous needle, osmotic pump, Polymer(s), but not limited to.

4. Molecular therapy, wherein comprises: drug delivery in a molecular state via: any said organisms to claim 1; further comprises molecular therapy via: skin, blood, or respiratory track, of any said organisms to claim 1, or a combination thereof, but not limited to; wherein said molecules to claim 1, are transferred via: said magnetic conductive recipient to claim 1, in turn via: said magnetized water to claim 1, but not limited to; in contact with any said organisms to claim 4, but not limited to.

5. Free radical extraction from said organisms to claim 1, executed via: said magnetized water to claim 1, but not limited to; in turn via: said magnetic conductive recipient to claim 1.

6. MRI (magnetic resonance imaging) achieved via: said magnetized water to claim 1, but not limited to; that is in physical contact with any said organisms to claim 1, in turn via: said magnetic conductive recipient to claim 1, therefore comprises: MWRI (magnetic water resonance imaging); remaining with the present embodiment,
other means for imaging further comprises: multiple channel imaging, via a transmitter receiver component via: said MWRI (magnetic water resonance imaging); wherein a signal is received via: a single channel (channel 1) of said transmitter receiver component to claim 6, that in turn is transmitted, via: a computer, via: a monitor (monitor 1); a second signal via: a MRI (magnetic resonance imaging), is received via: a different channel (channel 2), of said receiver transmitter component to claim 6, and transmitted via: said computer, via said (monitor 1), where signals from channel 1 and channel 2, are superimposed to form a single image; further comprises: programmed software to simulate processes to claim 6, of said receiver transmitter component(s) to claim 6, or a combination thereof, but not limited to.

7. Said magnetic conductive recipient to claim 1, as one or more doors, door retaining flange(s), water retaining seal(s), and/or an o-ring seal(s) immediately adjacent to said doors, but not limited to; further comprises: one or more hinges for opening or removing said door(s) via: lift-off latch hinges, quick disconnect latch hinges, well known to the art; means to retain doors shut include: pull-action toggle clamps, push/pull-action toggle clamps, door edge mount latches, door face-mount latches, door face mount latches with emergency release handles, well known to the art, but not limited to; conductive contact means are provided, between said door(s) to claim 7, and said magnetic conductive recipient to claim 7, via conductive material(s), placed along their immediate adjacent perimeters where contact is made, in a way as, not to hinder said water retaining seal(s) to claim 7.

8. Remaining with the present embodiment, said magnetic conductive recipient is to function in an open room environment, but not limited to; therefore comprises: a removable cover, handles, windows, window seals; air vents, water retaining seals, and/or an o-ring seal(s) found immediately adjacent to said cover, via: a grove along the top perimeter of said magnetic conductive recipient to claim 8, and said doors to claim 8, but not limited to; said cover as means to open, or be removed completely via: lift off latch hinges, quick disconnect latch hinges, well known to the art, but not limited to; said cover further comprises: pneumatic, shafts raising and lowering means, remote receiver transmitter signaling means; conductive contact means are provided between said cover, and said magnetic conductive recipient to claim 8, and said doors to claim 7 via: magnetic conductive material(s), placed underneath, and along the bottom perimeter area of said cover, and along the top perimeter of said magnetic conductive recipient to claim 8, and said doors to claim 8; said cover yet further comprises: means for light therapy, via: infra red lamp(s) ultra violet lamp(s), but not limited to; disinfection via: germicidal lamp(s), led lamp(s), lasers, but not limited to.

9. Remaining with the present embodiment, said magnetic conductive recipient to claim 1, said door(s) to claim 7, and said cover to claim 8, serves to activate and control said magnetized water to claim 1, via means described to claim 1; and are made of conductive and non conductive materials that comprises: wiring, superconductive wire, container layers (compartment layers), tubing (used to circulate carbon dioxide, within the structures), multiple layers, iron, steel, gold, platinum, silver, cadmium, palladium, radium, aluminum, titanium, metal plating, noble metal plating, polymers, refractive mirrors, superconductive mirrors, processors, electrodes, nanotubes, carbon nanotubes, graphene, polyetheretherketone, quantum sensors, silicone, magnets, electromagnets, magnetic field, water, magnetized water, super paramagnetic gels, responsive and non responsive gels (smart gels), static magnetism, ultrasound, silicon carbide, deuterated compounds (diamonds), lasers, refractive mirrors, gases, noble gases or a combination thereof, but not limited; said wiring, superconductive wire, said tubing, to claim 9, may be on the exterior, interior, or within the layers of said structures, or a combination thereof; non conductive materials comprises: glass, fiber glass, plastic, rubber, flexible or inflexible material(s), or a combination thereof, but not limited to;
materials, and means described to claim 9, serves as a pathway(s), via said magnetized water to claim 9, but not limited to, for said molecules to claim 1, to encompass, and attain said organism(s) to claim 1, but not limited to; the exterior said layer(s) to claim 9, of said magnetic conductive recipient to claim 9, provides a current barrier and/or magnetic field barrier, that comprises: aluminum, stainless steel, but not limited to, in order to minimize unwanted exterior sources of electric current, and/or magnetic fields from altering means, and processes, of the present embodiment.

10. Remaining with the present embodiment;
Said molecules to claim 4, derives from said substances to claim 1, but not limited to; said molecules to claim 4, are extracted from said substances to claim 1 via multiple existing means and methods that comprises: cloud point extraction, current charge, electric current, matter waves, quantum transport, quantum processing, air flow, ultrasound, pressure, polymers, vacuum (ultra high vacuum, high-vacuum thermal evaporation), ultra high speed gravitational extraction, polymeric gels, magnets, magnetic gels, super paramagnetic gels, ferrogels, antiferromagnetic gels, electromagnets, static magnetism, magnetic field, polyetheretherketone, lasers, electromagnetic hyperthermia,
turbulence, circulation, agitation, pulsation, steam H₂O distillation, solvent distillation, acetone distillation, hydrogen, carbon dioxide, Ion catchers (cryogenic noble gases), supercritical carbon dioxide extraction, molecular electronics (Rabi-assisted tunneling), photo activation (light-induced electron transfer), natural occurring positive, or negative current charge, induced positive or negative current charge, pulsation, agitation, magnetic field, high temperature magnetic field, static magnetism, electromagnetic hyperthermia, superconducting quantum interference device (SQUID) or a combination thereof, but not limited to; said molecules to claim 10, are then channeled via: said means described to claim 9, of said magnetic conductive recipient, doors, to claim 9, in turn via: said magnetized water to claim 9, but not limited to, that is in contact with, said organisms to claim 9; still remaining with the present embodiment; further comprises: said molecules to claim 10, being channeled via said cover to claim 9; yet further comprises: the use of gas forms: oxygen, hydrogen, but not limited to; substances in raw and/or liquid form, may be added to said magnetized water to claim 10, contained within said magnetic conductive recipient to claim 10, or a combination thereof, but not limited to.

11. Remaining within the spirit and scope of the present embodiment; comprises: means for therapeutic treatment, altering and controlling internal and/or external functions, of said cell organisms to claim 2, via:
a magnetic conductive recipient, having a removable magnetic conductive cover; conductive contact means provided via: the bottom surface of said magnetic conductive cover to claim 11, and the top surface of said magnetic conductive recipient to claim 11, that function as one unit when linked together;
said magnetic conductive recipient to claim 11, further comprises: a central cavity (depression, vial shape) within the micro or nano size, but not limited to; within said central cavity to claim 11, containing said magnetized water to claim 11; herein said cell(s), are placed (deposited), permitting control of said cell(s); via: said magnetized water to claim 11, that encircles said cell(s), over a (360 degrees range); in turn this permits directional control over North, South, East, West directions, in one or more directions simultaneously, including overlapping of the boundaries of said North, South, East, West directions; Consistent with Inverse Square Law
$$( \frac{S}{4 π {r}^{2}} = 1 ) ,$$
and achieved via the combined structures of said magnetic conductive recipient to claim 11, and said magnetic conductive cover to claim 11), in turn controlled via means described to claim 1, permitting control of internal and/or external functions of said cell(s) to claim 11, as described to claim 2, and control of internal and/or external functions of said mitochondrial to claim 2, to be altered, further comprises: control of internal speed of said cells and/or said mitochondrial functions, but not limited to; yet further comprises to claim 11: drug delivery via: said molecules to claim 10, via said structures to claim 11, in turn via means described to claim 1; yet still further comprises: drug delivery to claim 3, via: hypodermic needle, osmotic pump, polymer(s), but not limited to, and executed via: one or more tubes (multipurpose tubes) that run from the exterior of said cover to claim 11, via: the interior of said central cavity to claim 11;
said multipurpose tubes to claim 11, further serve as byways for executing: surgery, water replenishing, water overflow, escape valve(s), but not limited to;
said magnetic conductive cover to claim 11, serves to prevents evaporation, and/or contamination, of said magnetized water to claim 11;
said structures to claim 11, of said magnetic conductive recipient to claim 11, and said magnetic conductive cover to claim 11, is controlled and powered via: means described to claim 1, said structures to claim 11, comprises: conductive and non conductive materials, glass, metal, noble metals gold, platinum, silver, radium, cadmium, palladium, aluminum, titanium, metal plating, noble metal plating, polyetheretherketone, electromagnets, magnets, ferrogels, antiferromagnetic gels, superconductive mirrors, superconductive wire, graphene, nanotubes (carbon nanotubes); further comprises: electromagnetic components, Lasers, microprocessors, electrodes, polymers, sensors, superconductive sensors; methods comprising: pulsation, and/or via a combination of materials and methods thereof to claim 11, but not limited to; the exterior layer(s) of said magnetic conductive recipient to claim 11 yet further comprises:
a current barrier and/or magnetic field barrier, that comprises: aluminum, but not limited to, in order to minimize unwanted exterior sources of electric current, and/or magnetic fields from altering processes, to claim 11;
remaining with the present embodiment; further comprises: SQUID (superconductive quantum interference device), for ultra low frequency use (below 1 Telsa) via: said magnetic water, and for (MWRI) magnetized water resonance imaging to claim 6, and/or via a combination thereof, but not limited to.
